(19) United States
(12) Patent Application Publication
(10) Pub. No.: US 2018/0043011 A1
(43) Pub. Date: Feb. 15, 2018

(54) PI3-Kinase Inhibition and LAG-3 Checkpoint blockade as a Combination Therapy for Cancer

(71) Applicant: Wisconsin Alumni Research Foundation, Madison, WI (US)

(72) Inventors: Douglas G. McNeel, Madison, WI (US); Viswa T. Colluru, Salt Lake City, UT (US); Christopher D. Zahm, Madison, WI (US)

(21) Appl. No.: 15/674,066

(22) Filed: Aug. 10, 2017

Related U.S. Application Data
(60) Provisional application No. 62/372,967, filed on Aug. 10, 2016.

(51) Int. Cl. A61K 39/395; C07K 16/28; A61K 39/00; A61K 31/4745

(52) U.S. Cl. CPC A61K 39/3955; A61K 31/4745; C07K 16/2803; A61K 39/0011; A61K 2039/53

(57) ABSTRACT

This disclosure provides methods of treating cancer comprising administering at least one PI3K inhibitor and at least one LAG-3 checkpoint inhibitor. Suitable compositions are also disclosed.

### CROSS-REFERENCE TO RELATED APPLICATIONS

This application claims priority to U.S. Provisional Application No. 62/372,967 filed on Aug. 10, 2016, the contents of which are incorporated by reference in its entirety.

### STATEMENT REGARDING FEDERALLY SPONSORED RESEARCH

N/A

### BACKGROUND OF THE INVENTION

The field of the invention is regarding new combination treatment for cancer by regulating the immune response against cancer cells.

With the recent breakthrough clinical successes in cancer immunotherapies, even as single-agent therapies, a current major focus in the field of cancer research is discovery of rational targeted therapy and immunotherapy combinations. One predominant hypothesis is that tumor cells manage to evade dysfunctional T cells leading to tumor growth and cancer progression. Significant recent work has been done to characterize cell surface receptors in T cells that negatively regulate the function of T cells. Two of these receptors that are markers for ‘exhausted’ (dysfunctional) T cells are the programmed cell death protein 1 (PD1) and lymphocyte activation gene 3 (LAG-3) receptors. Work on PD1 has shown that tumor cells can evade T cells through PD1 signaling, and blocking the signaling can have a positive anti-tumor effect. PD1 signaling begins with binding to its ligand PDL1 or PDL2. Much of the therapeutic research in this field is focused on developing antibodies for PD1 or PDL1. PDL1 expression is controlled, in part, by the phosphoinositide 3-kinase (PI3K) signaling pathway providing another potential therapeutic strategy for affecting PD1 signaling.

A promising class of cancer-targeted agents inhibit PI3K activity to arrest tumor cell growth and maintenance. A PI3K inhibitor (Idelalisib) was approved by the FDA in 2014 for the treatment of chronic lymphocytic leukemia (CLL) and non-Hodgkin lymphoma (NHL).

Prostate cancer is a significant health risk for men over the age of 50, with about 200,000 newly diagnosed cases each year in the United States (Jemal A. et al., Cancer Statistics, 2005 (2005) CA Cancer J Clin, 55:10-30). It is the most common tumor diagnosed among men and the second leading cause of male cancer -related death in the United States (Jemal et al., Cancer Statistics, 2003 (2003) CA Cancer J Clin, 53:5-26). Despite advances in screening and early detection, approximately 30% of patients undergoing definitive prostatectomy or ablative radiation therapy will have recurrent disease at 10 years (Oefelein et al., 1997, J Urol, 158:1460-1465). While immune checkpoint blockades have gained traction in the treatment in many types of cancers, prostate cancer has been notoriously unaffected by this type of immunotherapy. There is a need for new treatments for prostate cancer, including metastatic disease, and new strategies are needed to eradicate microscopic disease to prevent the progression to clinically apparent metastasis.

Thus, there is a need for cancer treatments that can harness the subject's immune system response to treat, inhibit or prevent growth or metastasis of cancer, including prostate cancer.

### SUMMARY OF THE INVENTION

The present invention provides methods of treating cancer.

In one aspect, the disclosure provides a method of treating a subject having cancer, the method comprising administering to the subject at least one PI3K inhibitor and at least one LAG-3 checkpoint inhibitor, each in an amount effective, in combination, to treat the cancer.

In some aspects, the administering of the at least one PI3K inhibitor alters the cell surface biomarker profile of CD8+ cells from a mixed population of PD1^{high}LAG3^{low} and PD1^{high}LAG3^{med} to a majority of the CD8+ cells being PD1^{low}LAG3^{high} in the subject.

In another aspect, the method further comprises treating the subject with at least one additional immunotherapy or conventional therapy.

In some aspects, the LAG-3 checkpoint inhibitor is an anti-LAG-3 antibody. In some aspects, the cancer is prostate cancer, breast cancer or lung cancer.

In another aspect, the disclosure provides a method of targeting CD8+ T cells to reduce, inhibit or slow the growth of cancer cells or cancer metastasis in a subject, the method comprising administering at least one PI3K inhibitor and at least one LAG-3 checkpoint inhibitor in an amount effective to reduce, inhibit or slow the growth of cancer cells or cancer metastasis in the subject. In some aspects, the administering of the at least one PI3K inhibitor alters the phenotype on CD8+ cells from a mixed population of PD1^{high}LAG3^{low} and PD1^{high}LAG3^{med} to a majority of the CD8+ cells being PD1^{low}LAG3^{high} in the subject. In some aspects, the LAG-3 checkpoint inhibitor is an anti-LAG-3 antibody.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C demonstrate that PI3-Kinase inhibition results in increased LAG-3 and decreased PD-1 expression. FIG. 1A depicts WT (top) and OT1 (bottom) splenocytes stimulated with either CD3/28 beads or SIINFEKL peptide respectively, with/without PI3K inhibition and assayed for surface expression of LAG-3 (left graphs) or PD1 (right graphs) on activated (CD137+) CD8+ T cells at 24 h. FIG. 1B depicts a representative OT1 sample showing levels of LAG-3 and PD1 on CD8+ T cells upon in vitro SIINFEKL stimulation in the presence of a PI3K inhibitor. (* denotes a p-value <0.05, two way Mann-Whitney test) FIG. 1C depicts WT splenocytes stimulated with CD3/28 beads with/without PI3K inhibition and assayed for intracellular expression of cytokines in CD8+ T cells.

FIG. 2 shows representative images of (Left) mouse sarcoma cell line grown in normal media three days after plating shown at 4+ and 10+, and (Right) sarcoma cells from the same split plated with the same number of cells/cm² and grown in media containing the pan-PI3K inhibitor BEZ3.

### DETAILED DESCRIPTION OF THE INVENTION

The present disclosure provides methods of treating cancer. The disclosure further provides methods of reducing the number of cancer cells, inhibiting, retarding or slowing the growth of cancer cells, or inhibiting, retarding or slowing the metastasis of cancer cells in a subject having cancer. Also disclosed are methods of improving immunotherapy against cancer by modulating the PI3K pathway in combination with LAG-3 checkpoint inhibitor.

LAG-3 is a cell surface protein expressed on activated T cells that has been shown to be a marker for T cell dysfunction. The inventors have previously shown that LAG-3 -expressing CD8+ T cells produced through vaccination impaired tumor killing ability unless treatment is combined with LAG-3 -blocking antibodies. The present invention applies the effects of blocking LAG-3 on the immune cells in combination with at least one PI3K inhibitor to treat cancer. The combination of blocking both LAG-3 and PI3K with an inhibitor provides an improved immune-mediated anti-tumor response, resulting in the treatment of the cancer, as compared to the treatment with either LAG-3 inhibitor or PI3K inhibitor alone.

An “improved immune-mediated anti-tumor response” means an increase in the ability of one or more immune cells to recognize tumor cells. In some instances, the improved immune-mediated anti-tumor response results in an increased ability of one or more immune cells to target/recognize and kill cancer cells (e.g. CD8+ T cells). An improved immune-mediated anti-tumor response may be seen as a reduction in the number of cancer cells, inhibiting, retarding or slowing the growth of cancer cells, inhibiting, retarding or slowing the metastasis of cancer cells, increased infiltration of cytotoxic T cells into the tumor, or decreased inhibition of immune population within the tumor microenvironment

Not to be bound by any theory, the cell surface biomarker profile of CD8+ cells changes when PI3Kis inhibited from a mixed population of PD1^{high}LAG3^{med} and PD1^{high}LAG3^{low} to a majority of the CD8+ cells being PD1^{low}LAG3^{high}. This shift increases the effectiveness of anti-LAG-3 therapy to result in CD8+ T cells able to target cancer cells, reducing the number of cancer cells in the patient.

The terms “high,” “med” and “low” with reference to the cell surface biomarker profile of CD8+ cells (e.g. PD1^{high}LAG3^{low} and PD1^{high}LAG3^{med}) refer to the level of PD1 and LAG-3 surface markers on CD8+ T cells as measured by flow cytometry. “High,” “med” and “low” expression of the surface marker can be seen as a shift in the florescent intensity of the cell population (high expression has higher florescent intensity and low expression has lower intensity) measured using a fluorescently labeled antibody as compared to an unstimulated population of T cells. Low meaning a slight increase in expression over the unstimulated population, medium meaning a moderate increase, and high meaning a large increase in expression. Methods of determining the cell surface expression are known and understood by one skilled in the art.

In one embodiment, the present disclosure provides a method for treating a subject having cancer. The method comprises administering at least one PI3K inhibitor and at least one LAG-3 checkpoint inhibitor to a subject in an amount effective to treat the subject having cancer. In some embodiments, the method further comprises treating the subject with an additional immunotherapy or conventional therapy.

The terms “cancer,” “tumor” or “malignancy” are used throughout this description interchangeably and refer to the diseases of abnormal cell growth. Suitable cancer or tumors are known in the art. For example, but not limited to, suitable cancers include, prostate cancer, breast cancer, lung cancer, melanoma, renal cancer, bladder cancer, ovarian cancer, colorectal cancer, sarcomas such as osteosarcoma, renal cell carcinoma, haematopoietic and lymphoid malignancies and the like.

The term “treat” or “treatment” of cancer encompasses, but is not limited to, reducing, inhibiting or preventing the growth of cancer cells, reducing, inhibiting or preventing metastasis of the cancer cells or invasiveness of the cancer cells or metastasis or reducing, inhibiting or preventing one or more symptoms of the cancer or metastasis thereof

Phosphoinositide 3-kinase (“PI3K”) is an intracellular-signaling enzyme involved in a number of signaling pathways. PI3K catalyzes the production of phosphatidylinositol triphosphates, which drive the activation of Akt protein kinases. Akt in turn modulates the activity of numerous downstream signaling proteins, including the protein kinase mTOR. Activated PI3K signaling inhibits apoptosis and promotes cell growth, survival, and proliferation. While it is known that PI3K pathway activation plays a role in a number of cancers, the role of PI3K blockage in producing an anti-tumor immune effect is not known. Our studies have shown that PI3K inhibition alters the CD8+ T cell population, from a mixed population of PD1^{high}LAG3^{low} and PD1^{high}LAG3^{med} to a majority of the CD8+ cells being PD1^{low}LAG3^{high}.

Suitable PI3K inhibitors are known in the art and include, but are not limited to, for example, BKM120, BEX235, BGT226, Idelalisib, GDC-0941, IPI-145 (INK1197), GSK2636771, PI-103, among others. PI3K inhibitors may also be dual inhibitors that inhibit both mTOR and PI3K. Examples of such dual inhibitors include, but are not limited to, for example, BEZ235, BGT226, VS-5584m, (SB2343), PI-103, ZSTK474, GSK1059615, among others. PI3K inhibitors may also be subunit specific that inhibit single subunits of PI3K. Examples of such specific inhibitors include, but are not limited to, for example, p110α selective: Gedatolisib, HS-173, Alpelisib (BYL719), PIK-75, A66, YM201636; p110β selective: TGX-221, GSK2636771; p110γ selective: CZC24832, AS-252424, AS-604850, CAY10505; and p110δ selective: CAL-101 (Idelalisib, GS-1101), PIK-294, PI-3065, PIK-293, IC-87114 among others. The PI3K inhibitor is administered in pharmacologically acceptable compositions using suitable routes of administration and dosages as can be recognized and appreciated by one of ordinary skill in the art. In a preferred embodiment, the PI3K inhibitor is BEZ235. In another preferred embodiment, the PI3K inhibitor is a dual PI3K inhibitor and mTOR inhibitor.

Lymphocyte activation gene-3 (LAG-3; CD223) is a type I transmembrane protein expressed on the cell surface of activated CD4¹ and CD8¹ T cells and subsets of NK and dendritic cells (Triebel F, et al., J. Exp. Med. 1990; 171:1393-1405; Workman C J, et al., J. Immunol. 2009; 182(4): 1885-91). LAG-3 negatively regulates T cell signaling and functions. Blockade of LAG-3 can restore activities of the effector cells, diminish suppressor activity of T_{regs}, or enhance anti-tumor activity.

Suitable LAG-3 checkpoint inhibitors include, but are not limited to, for example, anti-LAG-3 antibody. Anti-LAG-3 antibodies are known in the art and commercially available, for example, BMS-986016 (Bristol-Myers Squibb). Suitable antibodies are also described in US Patent Publication No. 2015/0259420, the contents of which are incorporated by reference in their entirety. Other suitable LAG-3 checkpoint inhibitors include molecules that can prevent binding of LAG-3 to its ligands (e.g. major histocompatibility class II (MHC II) and/or Galectin-3) or molecules that inhibit signaling through the LAG-3 pathway. Not to be bound by any theory, but the combination of LAG-3 blockade with the PI3K inhibitor works better than the combination of PD-1 with a PI3K inhibitor due to the fact that PI3K inhibitors lower PD-1 expression on CD8+ T cells, making PD-1 blockade less effective. PI3K inhibitors also increase the expression of LAG3 on CD8+ T cells, making its blockade more effective.

The term “antibody” as used herein also includes an “antigen-binding portion” of an antibody. The term “antigen-binding portion,” as used herein, refers to one or more fragments of an antibody that retain the ability to specifically bind to an antigen (e.g., polypeptide or fragment thereof of LAG-3) and block signaling through the LAG-3 pathway. Examples of binding fragments encompassed within the term “antigen-binding portion” of an antibody include, but are not limited to, (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al, (1989) Nature 341:544-546), which consists of a VH domain; and (vi) an isolated complementarily determining region (CDR).

Antibodies used in the methods may be polyclonal or monoclonal; xenogeneic, allogeneic, or syngeneic; or modified forms thereof (e.g., humanized, chimeric, etc.). Suitable antibodies may be fully human or humanized. Preferably, antibodies of the invention bind specifically or substantially specifically to the antigen (e.g. LAG-3, polypeptides or fragments thereof). The term “monoclonal antibodies” refers to a population of antibody polypeptides that contain only one species of an antigen binding site capable of binding a particular epitope of an antigen, whereas the term “polyclonal antibodies” refers to a population of antibody polypeptides that contain multiple species of antigen binding sites capable of interacting with a particular antigen.

Suitable antibodies are able to inhibit or reduce at least one biological activity of the antigen (e.g. LAG-3) it binds. In certain embodiments, the antibodies or fragments thereof substantially or completely inhibit a given biological activity of the antigen.

The terms “subject suffering from cancer” or “subject having cancer” refer to a subject that presents one or more symptoms indicative of a cancer (e.g., a noticeable lump or mass) or metastasis thereof, or has been diagnosed after standard clinical investigation as having cancer or metastasis thereof

In some embodiments, the method of administering at least one PI3K inhibitor and at least one LAG-3 checkpoint inhibitor (e.g. anti-LAG-3 antibody) enhances the effect of an immunotherapy in a subject having cancer. Enhancement of immunotherapy may be the increased suppression, reduction or inhibition of cancer cell growth or metastasis as compared to the immunotherapy alone. In some embodiments, the method of administering at least one PI3K inhibitor and at least one LAG-3 checkpoint inhibitor (e.g. anti-LAG-3 antibody) is combined with at least one immunotherapy.

Immunotherapy as used herein is a therapy used to treat cancer by inducing, amplifying or enhancing an immune response against cancer cells. In some instances, immunotherapy may be a cell-based immunotherapy that employs target immune effector cells such as lymphocytes, macrophages, dendritic cells, natural killer cells (NK cells), cytotoxic T lymphocytes (CTL), and the like to target abnormal antigens expressed on the surface of cancer/tumor cells. In a preferred embodiment, at least one immunotherapy may be a T cell immunotherapy. Suitable T cell immunotherapies are known in the art and include, but are not limited to, for example, vaccines (e.g. DNA vaccine), oncolytic viral therapies that engage/recruit T cells, adoptive immunotherapies approaches (e.g. CAR T cells), or biospecific T cell engagers (BiTEs). Suitable vaccines include vaccines that result in the stimulation of effector cells. Suitable vaccines include, but are not limited to, for example, peptide, viral based, and tumor cell lysate. In some embodiments, the vaccine can be combined with the adoptive transfer of T cells (e.g. Adoptive Cell Therapy or ACT) specific for tumor antigens or the transfer of CAR T cells that are specific for a tumor antigen.

In a particular embodiment, the subject may suffer from prostate cancer. The immunotherapy may be a DNA vaccine targeted to prostate cancer. Suitable vaccines are known in the art and include, for example, a recombinant DNA vaccine that encodes the androgen receptor or fragments thereof or a peptide vaccine comprising a polypeptide androgen receptor or fragments thereof. Suitable recombinant DNA vaccines are disclosed in U.S. Pat. Nos. 7,910,565, 8,513,210 and 8,962,590, entitled “Prostate cancer vaccine,” and U.S. Pat. No. 7,179,797 and U.S. Application No. Ser. No. 11/615,778 entitled “Methods and compositions for treating prostate cancer using DNA vaccines” which are incorporated by reference in their entireties.

An “effective treatment” refers to treatment producing a beneficial effect, e.g., amelioration of at least one symptom of a cancer. A beneficial effect can take the form of an improvement over baseline, i.e., an improvement over a measurement or observation made prior to initiation of therapy according to the method. A beneficial effect can also take the form of reducing, inhibiting or preventing further growth of cancer cells, reducing, inhibiting or preventing metastasis of the cancer cells or invasiveness of the cancer cells or metastasis or reducing, alleviating, inhibiting or preventing one or more symptoms of the cancer or metastasis thereof. Such effective treatment may, e.g., reduce patient pain, reduce the size or number of cancer cells, may reduce or prevent metastasis of a cancer cell, or may slow cancer or metastatic cell growth. The terms “effective amount” or “therapeutically effective amount” refer to an amount sufficient to effect beneficial or desirable biological or clinical results. That result can be reducing, inhibiting or preventing the growth of cancer cells, reducing, inhibiting or preventing metastasis of the cancer cells or invasiveness of the cancer cells or metastasis, or reducing, alleviating, inhibiting or preventing one or more symptoms of the cancer or metastasis thereof, or any other desired alteration of a biological system.

The terms “metastasis” or “secondary tumor” refer to cancer cells that have spread to a secondary site, e.g., outside of the original primary cancer site. Secondary sites include, but are not limited to, for example, the lymphatic system, skin, distant organs (e.g., liver, stomach, pancreas, brain, etc.) and the like and will differ depending on the site of the primary tumor.

The terms “subject” and “patient” are used interchangeably and refer to any animal (e.g., a mammal), including, but not limited to, humans, non-human primates, rodents, and the like, which is to be the recipient of a particular treatment. Typically, the terms “subject” and “patient” are used interchangeably herein in reference to a mammalian, for example, human, subject.

The methods disclosed herein can include a conventional treatment regimen, which can be altered to include the steps of the methods described herein. The methods disclosed herein can include monitoring the patient to determine efficacy of treatment and further modifying the treatment in response to the monitoring. The methods disclosed herein can include administering a therapeutically effective amount of at least one PI3K inhibitor and at least one LAG-3 checkpoint inhibitor.

Conventional treatment regimens are known in the art and include, but are not limited to, surgical removal of the tumor, radiotherapy and chemotherapy. Conventional treatment regimens will differ depending on the type and stage of the cancer being treated and are known by one skilled in the art. For example, a suitable conventional treatment for prostate cancer includes ablation therapy alone or in combination with androgen deprivation therapy (ADT), second line androgen receptor pathway targeted agents, radiation therapy, or chemotherapy.

The administering of at least one PI3K inhibitor alters the phenotype on CD8⁺ cells from a mixed population of PD1^{high}LAG3^{low}, PD1^{high}LAG3^{med} and PD1^{low}LAG3^{high} to a majority of the CD8+ cells being PD1^{low}LAG3^{high}. A majority refers to the amount of CD8+cells which is at least 50% PD1^{low}LAG3^{high}. In some embodiments, a majority is at least 55%, alternatively at least 65%, alternatively at least 70%, alternatively at least 75%, alternatively at least 80%, alternatively at least 90%, alternatively at least 95% PD1^{low}LAG3^{high}, including any percentages in-between (e.g., at least 56%, 57%, 58%, 59%, 60%, 61%, 62%, 63%, 64%, 65%, 66%, 67%, 68%, 69%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, etc.). In a preferred embodiment, the majority is at least 85%, preferably at least 90% PD1^{low}LAG3^{high}.

Not to be bound by any theory, but by creating a more homogeneous CD8+ cell population expressing PD1^{low}LAG3^{high}, this T-cell population can subsequently be targeted by anti-LAG-3 antibody to de-repress the CD8+ T cells to identify and kill tumor cells.

In some embodiments, the administering of the at least one PI3K inhibitor alters the phenotype on CD8+ cells from a mixed population of PD1^{high}LAG3^{low}, PD1^{high}LAG3^{med} and PD1^{low}LAG3^{high} to a majority of the CD8+ cells being PD1^{low}LAG3^{high} in the subject.

In some embodiments, at least one PI3K inhibitor or at least one LAG3 checkpoint inhibitor may be formulated into a pharmaceutical composition. In some embodiments, both the at least one PI3K inhibitor and the at least one LAG3 checkpoint inhibitor are formulated into a single composition. In other embodiments the PI3K inhibitor and the LAG-3 checkpoint inhibitor are formulated into separate compositions that may be administered to the subject.

The PI3K inhibitor and LAG-3 checkpoint inhibitor may be administered simultaneously or sequentially. Simultaneous administration includes the administration of the PI3K inhibitor and LAG-3 checkpoint inhibitor in two different formulations, taken separately but within an hour of administration of the first inhibitor (e.g. seconds or minutes in-between). Suitably, when administered sequentially, for example, the PI3K inhibitor may be administered first followed by administration of the LAG-3 checkpoint inhibitor or the LAG-3 checkpoint inhibitor may be administered first followed by administration of the PI3K inhibitor. The time between the administration of the PI3K inhibitor and LAG-3 inhibitor can be adjusted for maximum efficacy, and may be in the order of minutes, hours, days or weeks.

The pharmaceutical compositions described herein may further include a pharmaceutically acceptable carrier. The term “pharmaceutically acceptable carrier” refers any carrier, diluent or excipient that is compatible with the other ingredients of the formulation and not deleterious to the recipient.

The at least one PI3K inhibitor or at least one LAG-3 checkpoint inhibitor may preferably be administered each with a pharmaceutically acceptable carrier selected on the basis of the selected route of administration and standard pharmaceutical practice for each inhibitor. The active agent may be formulated into dosage forms according to standard practices in the field of pharmaceutical preparations. See Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, injectable solutions, troches, suppositories, or suspensions. For antibodies, suitable dosages forms are normally solutions.

For oral administration, the active ingredient may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents.

For parenteral administration, the active agent may be mixed with a suitable carrier or diluent such as water, an oil (e.g., a vegetable oil), ethanol, saline solution (e, g., phosphate buffer saline or saline), aqueous dextrose (glucose) and related sugar solutions, glycerol, or a glycol such as propylene glycol or polyethylene glycol. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

The pharmaceutical composition is preferably in unit dosage form. In such form the preparation is divided into unit doses containing appropriate quantities of the active component.

In some embodiments, the PI3K inhibitor and the LAG-3 checkpoint inhibitor are respectively contained in separate compositions, these may be of the same dosage form or of different dosage forms. For example, the two may be mutually different dosage forms, each of which is one among oral formulation, parenteral formulation, injectable formulation, drip formulation, and intravenous drip formulation; or the two may be the same dosage form.

Suitable dosages of the PI3K inhibitor and the LAG-3 checkpoint inhibitor can be determined by one skilled in the art. In one embodiment, the dose of the PI3K inhibitor and the LAG-3 checkpoint inhibitor (e.g. anti-LAG-3 antibody) is calculated per mg/kg body weight. In another embodiment, the dose of the PI3K inhibitor and the LAG-3 checkpoint inhibitor is a flat- fixed dose.

It will be appreciated that appropriate dosages of the PI3K inhibitor or LAG3 checkpoint inhibitor, and compositions comprising a PI3K inhibitor or LAG3 checkpoint inhibitor, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, or materials used in combination, and the age, sex, weight, condition, general health, and prior medical history of the patient. The amount of compound and route of administration will ultimately be at the discretion of the physician. Administration in vivo can be effected in one dose, continuously or intermittently (e.g. in divided doses at appropriate intervals) throughout the course of treatment.

Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

For example, but not limited to, a suitable dosage range for the PI3K inhibitor may be from about 0.01 to about 1000 mg per day, such as, for example, from about 0.1 to about 800 mg per day, and from about 1 to about 500 mg per day.

For example, but not limited to, anti-LAG-3 antibody could be administered at dose of 0.2 mg/kg to about 2 mg/kg a day, for example, 0.4 mg/kg. In another embodiment, dosage regimens are adjusted to provide the optimum desired response (e.g., an effective response).

In some embodiments, methods of reducing, inhibiting or preventing cancer cell growth in a patient are provided. The method comprises administering an effective amount of the pharmaceutical compositions provided here, including, for example, a pharmaceutical composition comprising at least one PI3K inhibitor and at least one anti-LAG3 antibody, wherein the pharmaceutical composition is administered in an effective amount to reduce, inhibit or prevent cancer cell growth.

This disclosure also provides kits. The kits can be suitable for use in the methods described herein. Suitable kits include a kit for treating cancer comprising a pharmaceutical composition comprising at least PI3K and a pharmaceutical composition comprising at least one LAG-3 checkpoint inhibitor. In one aspect, the kit provides the pharmaceutical compositions in amounts effective for treating cancer. In some aspects, instructions on how to administer the pharmaceutical composition and/or active agents are provided.

In another embodiment, the disclosure provides a method of enhancing or increasing the CD8+ T cells response to reduce or inhibit cancer cell growth or metastasis in a subject, the method comprising administering at least one PI3K inhibitor and at least one LAG-3 checkpoint inhibitor (e.g. anti-LAG-3 antibody) in an amount effective to reduce tor inhibit cancer cell growth or metastasis in the subject.

In some embodiments, the present disclosure provides a method of altering T-cells destined to be dysfunctional to be functional. The method comprises administering at least one PI3K inhibitor and at least one LAG-3 checkpoint inhibitor (e.g. anti-LAG-3 antibody). The method alters the population of T-cell being activated by a therapy to express LAG-3 instead of a mixture of inhibitor receptors, allowing the additional blockade of LAG-3 resulting in active T-cell. This method results in the altered T CD8+ T cells to elicit an immune response and target tumor cells for death.

The present invention has been described in terms of one or more preferred embodiments, and it should be appreciated that many equivalents, alternatives, variations, and modifications, aside from those expressly stated, are possible and within the scope of the invention.

The following non-limiting examples are included for purposes of illustration only, and are not intended to limit the scope of the range of techniques and protocols in which the compositions and methods of the present invention may find utility, as will be appreciated by one of skill in the art and can be readily implemented.

### EXAMPLES

### Example 1

### PI3K Shifts the Phenotype on CD8+ Cells In Vitro

PI3K inhibition (PI3ki) is an effective therapy option for human malignancies¹. In addition, immune checkpoint blockade is gaining traction in many types of human cancers; unfortunately, prostate cancer has been notoriously unaffected by this novel immunotherapy. The goal of a successful cancer immunotherapy is to generate tumor-unregulated cytotoxic CD8⁺ T-cells. With that goal in mind, our data demonstrates that stimulating these T-cells in the presence of a pan-PI3Ki (BEZ235) skews their regulation down one specific pathway instead of at least two distinct pathways, namely the LAG-3 pathway instead of both PD1 and LAG-3 . PI3Ki results in decreased expression of other regulatory receptors and increased expression of LAG-3; a regulatory receptor that has been shown to be detrimental to a productive anti-tumor immune response. (See, e.g., Grosso J F, Kelleher C C, Harris T J, Maris C H, Hipkiss E L, De Marzo A, Anders R, Netto G, Getnet D, Bruno T C, et al. LAG-3 regulates CD8+ T cell accumulation and effector function in murine self- and tumor-tolerance systems. Journal of Clinical Investigation 2007; 117:3383-92). Thus, combination therapy containing LAG-3 blockade and PI3Ki is promising and we believe the two show a synergistic anti-tumor effect that will render blockade of multiple checkpoint proteins unnecessary. In addition, BEZ235 treatment results in reduced expression of PD-1, the most commonly targeted immune checkpoint receptor, suggesting that PI3Ki will render PD-1 blockade less effective when compared to a PD-1 monotherapy. While other groups are combining PI3K inhibitor with PD1 blockade, since both have been shown to have effective anti-tumor properties, the experiments presented here demonstrate that that combination (PI3K inhibitor and PD-1) would not be expected to work well in combination. While anti-PD1 monotherapy has been shown to be more effective against tumors than anti-LAG3 to date, you would not necessary add an inferior therapy, e.g. LAG-3 inhibitor, with PI3K inhibition. However, given our data shown in the examples below, we expect our combination of anti-LAG3 and PI-3K to work better than PD1 blockade.

### In Vitro Stimulation

Splenocytes from either OT-1 or wild type (WT) C57BL/6J mice were cultured in RPMI-1640, 10% FCS, 200U/mL Pen/Strep, 1% NaPyr, 1% HEPES, 50 μM β-MeOH and 2 μg/mL SIINFEKL peptide (OT1 cells) or CD3 and CD28 antibody coated latex beads (WT cells) with or without 200nM BEZ235 PI3-Kinase inhibitor (Selleckchem, Catalog No. S1009).

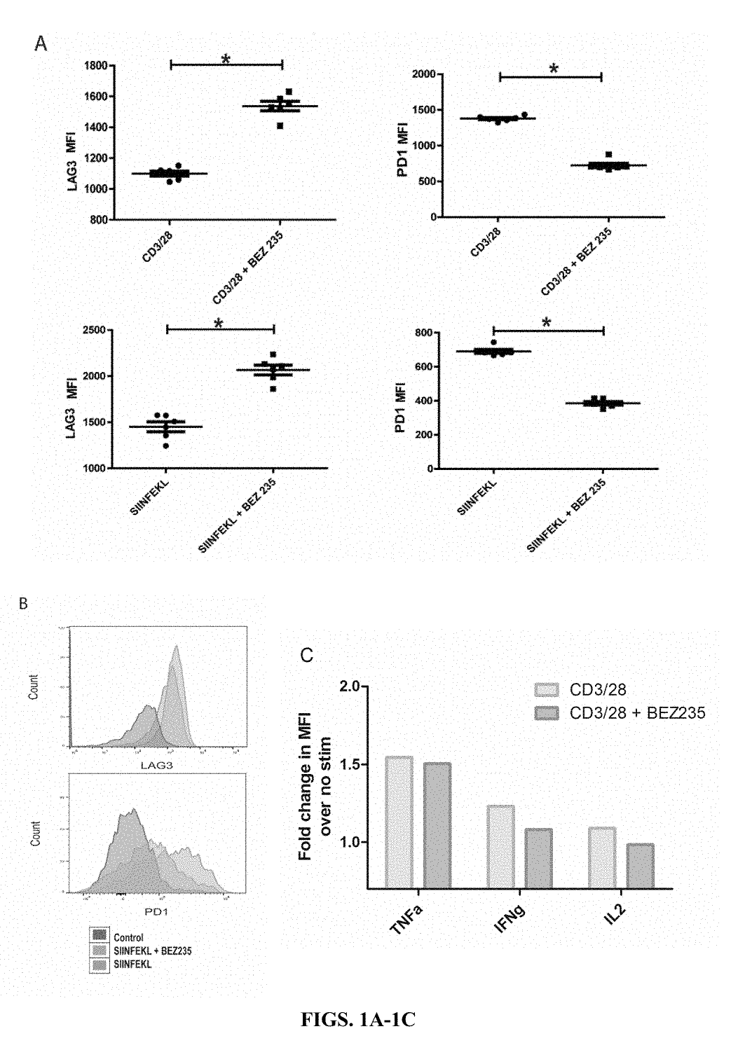

FIG. 1A shows wildtype and OT1 splenocytes stimulated with either CD3/28 beads or SIINFEKL peptide respectively, with/without PI3K inhibition and assayed for surface expression of LAG3 (left graphs) or PD1 (right graphs) on activated (CD137+) CD8+ T cells at 24 h. FIG. 1B shows a representative analysis of an OT1 sample showing levels of LAG3 and PD1 upon in vitro SIINFEKL stimulation in the presence of a PI3K inhibitor. (* denotes a p-value <0.05, two way Mann-Whitney test). FIG. 1C shows the analysis of intracellular cytokine levels within CD8+ T cells taken from wildtype splenocytes that were stimulated with CD3/28 beads with/without PI3K inhibition. These data demonstrate that PI3K inhibition does not reduce the functional activation CD8+ T-cells.

### Example 2

### PI3Ki Prevent Cancer Cell Growth In Vitro

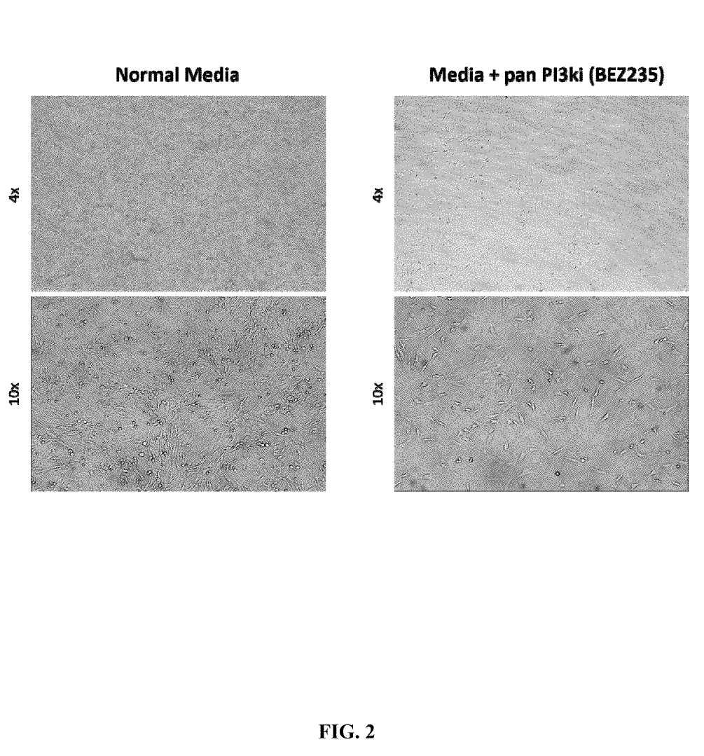

An SSX2 mouse sarcoma cell line (described in Rekoske et al., Cancer Immunol Res August 2015 3; 946) was split and 100,000 cells plated into each of two 75 mm² flasks. Cells were grown in RPMI-1640, 5% FCS, 200 U/mL Pen/Strep, 50 μM β-MeOH with or without 200 nM BEZ235 PI3-Kinase inhibitor. Images of the cells after three days are shown in FIG. 2. FIG. 2 left, mouse sarcoma cell line grown in normal media three days after plating; shown at 4+ and 10× and right, sarcoma cells from the same split plated with the same number of cells/cm² and grown in media containing the pan-PI3K inhibitor BEZ235. BEZ235 inhibited cell growth.

### Example 3

### Testing of Alternative PI3Ki

The pan-PI3Ki BEZ235, used in FIGS. 1 and 2, targets the p110α, p110β, p110δ, and p110γ subunits of PI3K as well as the mTOR kinase domain p10S6K, inhibiting multiple molecules involved in the PI3K/Akt/mTOR pathway. It has also been shown to inhibit FLT3, JAK2, PDK-1, CDK1, and B-Raf Other more specific PI3Kis that have recently been developed are tested to determine if the CD8+ T cell shift effect seen with BEZ235 is specific to PI3K or if it requires the inhibition of these other pathways as well. Thus the following specific PI3K inhibitors are tested: AS-605240, PIK-90, HS-173 AS-605240 and PIK-90 (Selleckchem Catalog No S1410 and S1187 respectively) selectively inhibiting PI3K with no off target inhibition. In addition, many selective PI3Kis have been developed to target single subunits of PI3K. For example, the p110α subunit is known to have greater activity in T-cells when compared to the other three; so a molecule like HS-173 (Selleckchem Catalog No.S7356) which specifically targets p110α is of interest. After testing deferent types of PI3Kis we can select for inhibitors that skew the T-cells toward the highest LAG-3, lowest PD1 phenotype to use in anti-tumor studies.

### Example 4

### Anti-Tumor Studies

Anti-tumor studies are conducted in a subcutaneous murine tumor model and treatments will include various selected PI3Kis alone or in combination with either LAG-3 or PD1 blockade. These experiments determine if there is a synergistic effect when PI3Ki is used in combination with LAG-3 immune checkpoint blockade. They provide information about the biology underlying any increase in efficacy observed by allowing us to determine if the PI3Ki induced altered phenotype renders CD8⁺ T-cells more susceptible to immune checkpoint therapy; if the effects are specific to a certain PI3Ki and if it is the increase in LAG-3 that is responsible for any increased therapeutic efficacy.

Relevant Literature Links:
1. Maira, S-M (2011). PI3K Inhibitors for Cancer Treatment: Five Years of Preclinical and Clinical Research after BEZ235. Mol Cancer Ther 10: 2016-2016.
2. Stark, A-K, Sriskantharajah, S, Hessel, E M and Okkenhaug, K (2015). PI3K inhibitors in inflammation, autoimmunity and cancer. Current Opinion in Pharmacology 23: 82-91.
3. Kim, T, Amaria, RN, Spencer, C, Reuben, A, Cooper, Z A and Wargo, J A (2014). Combining targeted therapy and immune checkpoint inhibitors in the treatment of metastatic melanoma. Cancer Biol Med 11: 237-246.
4. Voskoboynik, M, Arkenau, H-T, Voskoboynik, M and Arkenau, H-T (2014). Combination Therapies for the Treatment of Advanced Melanoma: A Review of Current Evidence, Combination Therapies for the Treatment of Advanced Melanoma: A Review of Current Evidence. Biochemistry Research International, Biochemistry Research International 2014, 2014: e307059.
5. Pardoll, D M (2012). The blockade of immune checkpoints in cancer immunotherapy. Nat Rev Cancer 12: 252-264.

1. A method of treating a subject having cancer, the method comprising:
administering at least one PI3K inhibitor and at least one LAG-3 checkpoint inhibitor an each in an amount effective, in combination, to treat the cancer.

2. The method of claim 1, wherein the administering of the at least one PI3K inhibitors alters the phenotype on CD8+ cells in the subject from a mixed population of PD1^{high}LAG3^{low} and PD1^{high}LAG3^{med} to a majority of the CD8+ cells being PD1^{low}LAG3^{high}.

3. The method of claim 1, wherein the PI3K inhibitor is selected from the group consisting of BKM120, BEX235, BGT226, Idelalisib, GDC-0941, IPI-145 (INK1197), GSK2636771, PI-103, VS-5584, (SB2343), PI-103, ZSTK474, GSK1059615 and a combination of any two or more.

4. The method of claim 3, wherein the PI3K inhibitor is BEZ235.

5. The method of claim 1, wherein the at least one LAG-3 checkpoint inhibitor is an anti-LAG-3 antibody.

6. The method of claim 1, wherein the method further comprises treating the subject with at least one additional immunotherapy.

7. The method of claim 6, wherein the at least one additional immunotherapy is a DNA vaccine.

8. The method of claim 1, wherein the subject is a mammal.

9. The method of claim 1, wherein the subject is a human.

10. The method of claim 1, wherein the cancer is selected from the group consisting of prostate cancer, breast cancer, and lung cancer.

11. The method of claim 8, wherein the cancer is prostate cancer.

12. A method of targeting CD8+ T cells to reduce or inhibit cancer cell growth or metastasis in a subject, the method comprising administering at least one PI3K inhibitor and at least one LAG-3 checkpoint inhibitor each in an amount effective, in combination, to reduce or inhibit cancer cell growth or metastasis in the subject.

13. The method of claim 12, wherein the administering of the at least one PI3K inhibitors alters the phenotype on CD8+ cells in the subject from a mixed population of PD1^{high}LAG3^{low} and PD1^{low}LAG3^{high} to a majority of the CD8+ cells being PD1^{low}LAG3^{high} in the subject.

14. The method of claim 13, wherein the PI3K inhibitor is selected from the group consisting of BKM120, BEX235, BGT226, Idelalisib, GDC-0941, IPI-145 (INK1197), GSK2636771, PI-103, VS-5584, (SB2343), PI-103, ZSTK474, GSK1059615

15. The method of claim 14, wherein the PI3K inhibitor is BEZ235.

16. The method of claim 12, wherein the LAG-3 checkpoint inhibitor is an anti-LAG3 antibody.

17. The method of claim 12, wherein the method further comprises treating the patient with at least one immunotherapy.

18. The method of claim 17, wherein the immunotherapy is a DNA vaccine.

19. The method of claim 12, wherein the subject is a mammal.

20. The method of claim 19, wherein the subject is a human.

21. The method of claim 12, wherein the cancer is selected from the group consisting of prostate cancer, breast cancer, lung cancer, melanoma, renal cancer, bladder cancer, ovarian cancer, colorectal cancer, sarcomas such as osteosarcoma, renal cell carcinoma, and hematopoietic and lymphoid malignancies.

22. The method of claim 21, wherein the cancer is prostate cancer.

23. The method of claim 1, wherein the method further comprises at least one conventional cancer therapy.

24. The method of claim 23, wherein the conventional cancer therapy is chemotherapy or radiation treatment.

25. A pharmaceutical composition for treatment of cancer comprising at least one PI3K inhibitor and at least one LAG-3 checkpoint inhibitor each in an amount effective, in combination, to treat cancer.

26. The pharmaceutical composition of claim 25, wherein the PI3K inhibitor is BEZ235.

27. The pharmaceutical composition of claim 26, herein the LAG-3 checkpoint inhibitor is an anti-LAG-3 antibody.
